(19) United States
(12) Plant Patent Application Publication
(10) Pub. No.: US 2005/0022276 P1
(43) Pub. Date: Jan. 27, 2005

(54) Grapevine denominated "Margaux"

(50) VITIS VINIFERA; MARGAUX

(76) Inventor: Joseph Maranto, Bakersfield, CA (US)

Correspondence Address: WORREL & WORREL;ST. CROIX PROFESSIONAL CENTER, SUITE 121, 2109 W. BULLARD AVE., FRESNO, CA, 93711-1258, US

(21) Appl. No.: 10/624,709

(22) Filed: Jul. 21, 2003

(51) Int. Cl. A01H 5/00

(52) U.S. Cl. PLT/207

(57) ABSTRACT

A new and distinct variety of grapevine which is distinguished by its productivity bearing fruit in mid to late October in the San Joaquin Valley of central California in compact clusters having large seedless berries of olive green skin coloration and a firm, crisp flesh and which is a cross of the “Red Globe” and “Crimson” grapevines, but from which it is distinguished in a number of respects including the foregoing.

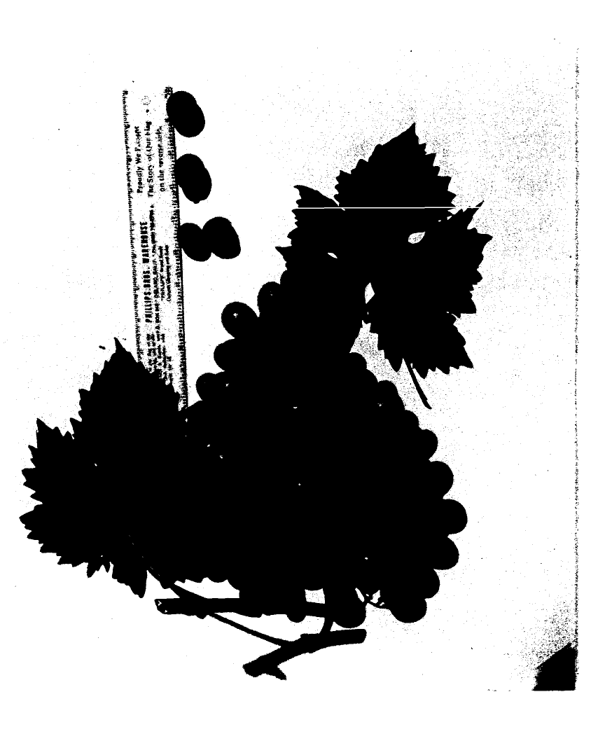

### CROSS-REFERENCE TO RELATED APPLICATIONS

Not applicable.

### STATEMENT REGARDING FEDERALLY SPONSORED RESEARCH OR DEVELOPMENT

Not Applicable.

### BACKGROUND OF THE NEW VARIETY

The present invention relates to a new and distinct variety of grapevine, which will hereinafter be denominated varietally as the “MARGAUX” grapevine, and, more particularly, to a grapevine which is vigorous and productive and which produces large and very firm, generally olive green colored, fruit and which is mature for commercial harvesting and shipment in mid to late October in the San Joaquin Valley of central California.

The development of new varieties of fruit bearing plant life is fraught with concerns which must be successfully confronted for such new varieties to achieve commercial success. This is true of both tree and vine varieties wherein there are, in each case, a multitude of existing varieties.

In the case of grapevines, there is such a plethora of preexisting varieties that any of a host of possible distinguishing characteristics may be of substantial commercial consequence for new varieties which are developed. Such characteristics as vigor, productivity, resistance to disease and pests in the grapevines themselves are important. Such considerations as size, coloration, flavor, whether seedless or not, and a great number of other characteristics of the fruit of the new varieties are also important. These distinguishing characteristics have a direct bearing upon the decision as to whether or not to proceed with the commercial development of a new variety of grapevine.

The new variety of grapevine of the present invention evidences considerable promise in a number of its distinguishing characteristics, as hereinafter set forth in detail.

### ORIGIN AND ASEXUAL REPRODUCTION OF THE NEW VARIETY

The present variety of grapevine, Vitis vinifera, hereof was discovered by the inventor in a grapevine breeding project initiated in 1992 in a vineyard which is located near Delano, in the San Joaquin Valley of central California. The grapevine of the new variety was discovered by the inventor among an innumerable number of cross combinations in the breeding project. The breeding project produced several thousand test seedlings which were evaluated for their characteristics as to a multiplicity of considerations including, but not limited to fruitfulness, seedlessness and high quality as a table grape variety. The grapevine was discovered in a cross of the “Red Globe” grapevine, which was the female parent, and the “Crimson” grapevine, which was the male parent. Throughout the growing season, the variety of the present invention continued to be distinguishable from any known grapevine varieties. Of particular note, the fruit of the new variety consistently maintained the date of maturity hereinafter set forth and clusters of grapes having firm berries. In summary, the present variety is substantially distinguishable from its parentage and has very distinctive quality characteristics. Especially noticeable in the new variety are the very hard berries of green olive coloration having an outstanding size for a seedless grape.

In 1998, the present variety was asexually reproduced by the inventor by being successfully grafted on mature “Thompson Seedless” grapevine root stock in the same vineyard of discovery located near Delano in the San Joaquin Valley of central California. The asexually reproduced grapevines of the instant variety were carefully observed by the inventor in 1999 and 2000 and subsequently were confirmed in all respects to be identical to the original grapevine of the new variety.

### SUMMARY OF THE NEW VARIETY

The grapevine denominated “MARGAUX” is characterized by producing large seedless berries which have an olive green coloration and are ripe for commercial harvesting and shipment approximately in mid to late October in the San Joaquin Valley of central California. The new variety does not resemble any other known varieties and is distinguishable therefrom in a number of respects including the aforementioned ripening date. The fruit is seedless having a sweet flavor, a long storage life and a good eating quality.

### BRIEF DESCRIPTION OF THE DRAWING

The drawing is a color photograph of representative portions of the new variety showing a cluster of grapes thereof with an attached section of vine and foliage revealing the underside thereof, representative foliage revealing the upperside thereof, a berry sectioned transversely and laid open to disclose the flesh thereof and a berry sectioned longitudinally and laid open to display the flesh thereof.

### DETAILED DESCRIPTION

Referring more specifically to the botanical details of this new and distinct variety of grapevine, the following has been observed under the ecological conditions prevailing at the vineyard of origin which is located near Delano in the San Joaquin Valley of central California. All major color code designations are by reference to the Dictionary of Color, by Maerz and Paul. Common color designations are also occasionally employed.

### VINE

Generally:
   Size.—Large.
   Vigor.—Vigorous.
   Productive capacity.—Productive.
   Trunk.—Size — Slender. Very long split straps.
   Color.—Bark — Dark brown (8-E-11) — Underbark — Medium brown (7-C-12) India Tan.
   Canes.—Medium length 5′ (152 cm) to 10′ (304 cm).
   Thickness.—Medium. Width at node is 0.5 inch (12.7 mm).
   Nodes.—Round. Length between the nodes is 4 inches (101.6 mm) to 5 inches (127 mm).
   Shoots.—Length — Medium to long.
   Shoots.—Shape — circular to slightly flat.
   Shoots.—Contour — Smooth.
   Growing tip.—Generally straight up.
   Tendrils.—Length — 4 inches (101.6 mm) to 6 inches (152.4 mm).
   Tendrils.—Location — Discontinuous.
   Tendrils.—Form — Bifurcated and trifurcated.
   Tendrils.—Texture — Smooth.
   Buds.—Shape — Pointed.
   Buds.—Size — Medium.
   Bud break.—Near Delano in the San Joaquin Valley of central California, bud break is at the end of March.

### LEAVES

Size.—Generally — Medium to large.
Density.—Heavy.
Average length.—Mature Leaf — 4 inches (101.6 mm) to 5 inches (127 mm).
Color.—Dorsal surface — Green (22-J-8).
Color.—Ventral surface — Pale green (22-J-5) — Leek green.
Texture.—Upper Surface — Smooth.
Texture.—Lower Surface — Glabrous.
Petiole.—Length — 4 inches (101.6 mm) to 5 inches (127 mm).
Petiole.—Shape — Mostly round.
Petiole sinus.—Form — U shaped, lateral sinus V shaped, overlapping.
Lobe.—Mostly five pointed.
Color.—Leaf Vein — Midrib — Grape green (21-J-1).
Marginal form.—Generally — Serrate, average 12 pointed per lobe.
Teeth.—Size — Irregular.
Teeth.—Number — 50 to 60.

### FLOWERS

Date of bloom.—May 10.
Date of full bloom.—May 15.
Type.—Fertile.
Pistils.—Color — Green (22-L-5) Cerro green.
Amount of pollen.—Abundant.

### FRUIT

Maturity when described: Ripe for commercial harvesting and shipment approximately mid to late October near Delano in the San Joaquin Valley of Central California, about four to five weeks later than the “Red Globe” grapevine and “Crimson” grapevme.
   Solids — Sugar — 18.2%.
   Acid.—0.382.
   Sugar/Acid ratio.—47.3.
   Juice pH.—3.90.
   Seeds.—None, seedless.
   Capstem.—Strong.
Size:
   Cluster.—Generally — Medium to large.
   Cluster.—Weight — Average one pound.
   Compactness.—Compact.
   Cluster.—Form — Conical shouldered.
   Berry.—Size — Medium to large.
   Berry.—Form — Uniform.
   Dimensions.—Along Longitudinal Axis — 0.8 inch (20 mm) to 1 inch (25 mm).
   Dimensions.—Along Transverse Axis — 0.52 inch (13 mm) to 0.6 inch (15 mm).
   Pedicel.—0.32 inch (8 mm) to 0.4 inch (10 mm).
   Berry.—Numbers — 110 to 140 per cluster.
   Berry.—Weight — 0.21 ounces (6 grams) to 0.245 ounces (7 grams).
   Form.—Ellipsoidal.
Skin:
   Thickness.—Thick.
   Texture.—Very Firm.
   Tendency to crack.—None.
   Color.—Olive green (15-L-4) and some amber lime yellow (11-L-5) on sun exposed berries.
   Pulp.—Adheres to skin — olive green.
   Lenticels.—A few round pore like spots.
Flesh:
   Flesh color.—Colorless.
   Flavor.—Sweet, crisp.
   Eating quality.—Good.
Use: Dessert.
Keeping quality: After two months in the storage, still in good appearance.
Resistance to disease: No disease visible.
Resistance: Resistant to cold, drought, heat and wind.
Shipping and handling qualities: Excellent.

Although the new variety of grapevine possesses the described characteristics noted above as a result of the growing conditions prevailing near Delano, Calif., in the central part of the San Joaquin Valley of California, it is to be understood that variations of the usual magnitude and characteristics incident to changes in growing conditions, irrigation, fertilization, pruning, pest control, climatic variations and the like are to be expected.

1. A new and distinct variety of grapevine substantially as illustrated and described which is distinguished by producing seedless fruit which are mature for commercial harvesting and shipment approximately in mid to late October in the San Joaquin Valley of central California, which is vigorous and productive having compact and attractive clusters, with very firm large berries having a generally olive green skin coloration.
